(19) United States
(12) Patent Application Publication
(10) Pub. No.: US 2014/0267330 A1
(43) Pub. Date: Sep. 18, 2014

(54) SYSTEMS AND METHODS FOR MANAGING MEDICAL IMAGE DATA FOR MULTIPLE USERS

(71) Applicant: VOLCANO CORPORATION, San Diego, CA (US)

(72) Inventor: Jason Sproul, San Diego, CA (US)

(73) Assignee: VOLCANO CORPORATION, San Diego, CA (US)

(21) Appl. No.: 14/209,753

(22) Filed: Mar. 13, 2014

Related U.S. Application Data
(60) Provisional application No. 61/784,524, filed on Mar. 14, 2013.

(51) Int. Cl. G06F 3/14; A61B 5/02

(52) U.S. Cl. CPC G06F 3/1423; A61B 5/02007
USPC 345/522

(57) ABSTRACT

The invention provides systems and methods that provide a plurality of different displays (i.e., data formats) corresponding to intravascular imaging, such as obtained with intravascular ultrasound (IVUS) or optical coherence tomography (OCT). The plurality of displays may be provided to a single user, e.g., a cardiovascular surgeon, or the displays may be divided between multiple users, e.g., a surgeon, a surgical tech, and a radiologist.

### RELATED APPLICATIONS

This application claims priority to, and the benefit of, U.S. Provisional Application No. 61/784,524, filed Mar. 14, 2013 and incorporated by reference herein in its entirety.

### FIELD OF THE INVENTION

The invention generally relates to systems and methods for managing medical image data for multiple users.

### BACKGROUND

The number of interventional cardiovascular procedures performed each year continues to grow as more Americans suffer from cardiovascular ailments while the number of doctors trained in the relevant skills also increases. In 2008, for example, approximately 6.5 million diagnostic and therapeutic interventional procedures were performed, with the majority of them involving one or more intravascular entries. See MedTech Insight, “U.S. Markets for Interventional Cardiology Products—February 2010.” The procedures span a huge range of complexity from simple angioplasty to intravascular heart valve replacement. Many of these procedures are performed concurrently with intravascular imaging because external imaging (i.e., MRI, ultrasound) does not provide sufficient detail to evaluate the condition of a vessel, valve, aneurism, etc.

Current intravascular imaging systems use a serialized step-by-step process for acquiring and utilizing imaging information. Typically, clinical users first acquire images of a vessel segment using an intravascular modality such as ultrasound (IVUS) or optical coherence tomography (OCT). Once the images are acquired, they are processed for analysis by a physician or other clinical staff to determine whether and how to treat the patient. For example, after reviewing the images, a provided might remove the imaging device and performing treatment with an angioplasty catheter, or refer the patient to another specialist for more invasive treatment. In many cases, the images are determinative of the standard of care, for example the size or weight of a stent that is deployed.

Because of the serial nature of the intravascular imaging, the imaging process can become a bottle neck to providing more treatment, or to treating more patients in a given time period. For example, a patient with complex health issues cannot be sedated for long periods of time. If a provider must interrupt a procedure to evaluate image data, it is possible that the provider will not have adequate time to deliver all therapeutic care that would otherwise be possible during the sedation. Accordingly, the patient will have to return for additional procedures, thereby increasing the costs associated with that patient's care. Additionally, time lost reviewing imaging data translates into lost revenue for the treatment facility because fewer procedures can be performed per year. In areas without sufficient cardiovascular expertise, time lost reviewing imaging data may mean that few patients have access to well-trained cardiovascular surgeons.

### SUMMARY

The invention improves the efficiency of the intravascular intervention procedure by allowing users to perform measurements and other analyses simultaneously as imaging data is collected. Because multiple users can interact with the images simultaneously through separate interfaces, the “correct” clinical conclusion can be resolved faster. The system also reduces the procedure time, and physical stress on the patient, while providing more resources for the clinical team. Aspects of the invention are accomplished by a system that includes a central processing unit (CPU), and storage coupled to the CPU for storing instructions. The stored instructions, when executed by the CPU, cause the CPU to accept as input, real-time image data representative of an inside of a lumen from an intravascular imaging device. The CPU is additionally caused to associate the data with the type of device used to acquire the data. The CPU is also caused to process the data into a plurality of different displays. The CPU is further caused to determine which user should see which type of display, and provide as an output, the proper display to each user.

The data may be processed into any number of different displays, such as two, three, four, five, 10, etc. displays. In an exemplary embodiment, there are three types of displays. Those displays include real-time image display; image display at a fixed rate; and a paused image. Typically, the paused image may be used to make analytical measurements about the lumen, e.g., the free luminal area.

Systems and methods of the invention are configured such that multiple users may be provided a display simultaneously. Additionally, one or more users may be provided more than one display. In an embodiments, the system prevents a user from seeing a specific type of display. For example, in certain medical procedures, an operator in an operating room is prevented from seeing a real-time display.

Systems and methods of the invention may accept data from any intravascular imaging device. Exemplary devices include intravascular ultrasound (IVUS) devices and an optical coherence tomography (OCT) devices. With such devices, the data accepted by the system is IVUS data or OCT data. Alternative modalities such as visible or spectrographic imaging may also be used.

Systems of the invention may also have additional functionality. For example, systems of the invention may provide instructions such that the CPU is further caused to textually label the type of data to be displayed. Systems of the invention may provide additional instructions such that the CPU is further caused to color-code the image data or the background over which the image is displayed.

Another aspect of the invention provides methods for managing medical image data for multiple users. Methods of the invention involve receiving in real-time, image data representative of an inside of a lumen from an intravascular imaging device, associating the data with the type of device used to acquire the data, processing the data into a plurality of different displays, determining which user should see which type of display, and providing as an output, the proper display to each user.

### BRIEF DESCRIPTION OF THE DRAWINGS

FIG. 1 illustrates a timeline view of simultaneous operation;

FIG. 2 illustrates an exemplary user interface, such as may be found in an intravascular catheter laboratory. The interface of FIG. 2 provides multiple displays to a single user;

FIG. 3 illustrates a user interface that may be seen by a user not present in the catheter lab. The user interface may additionally distinguish real-time displays from reduced-rate displays;

FIG. 4 shows a system for executing the methods of the invention over a distributed network.

### DETAILED DESCRIPTION

The invention generally relates to systems and methods for managing medical image data for multiple users. Systems of the invention include a central processing unit (CPU), and storage coupled to the CPU for storing instructions. The stored instructions, when executed by the CPU, cause the CPU to accept as input, real-time image data representative of an inside of a lumen from an intravascular imaging device. The CPU is additionally caused to associate the data with the type of device used to acquire the data. The CPU is additionally caused to process the data into a plurality of different displays. The CPU is further caused to determine which user should see which type of display, and provide as an output, the proper display to each user.

The present invention involves providing the user with an interface or set of interfaces intended to facilitate simultaneous operation. FIG. 1 illustrates a timeline view of simultaneous operation. Typically, once sufficient data has been acquired by the imaging modality, the image is displayed in one or more formats, allowing a user to analyze the data. Because the user performing the procedure may be occupied with other tasks, such as guiding the imaging device or viewing an angiogram, the invention allows another user to evaluate the data in near real-time.

The data collected with the imaging modality will typically be available to the user performing the procedure. As shown in FIG. 2, the physician user may interact with a handheld unit and catheter to control the workflow and acquisition of images. However, as discussed with respect to FIG. 1, another user may conduct measurements on a selected frame from the image data which has been captured thus far. The invention is not limited to a handheld or computer monitor display, however, as the invention can make use of touch screens, voice recognition, goggles, or specialty interfaces, such as GOOGLE GLASS™.

In some embodiments, a second user can affect the views that are shown to the user conducting the procedure. For example, the second user can navigate through the images already acquired even as new data is arriving, as shown by the downward arrows below the image data in FIG. 2. The start and end control triangles and current tomographic frame control line exemplify means for the user to control display of images individually or as a loop. Additional related controls may be provided in another area of the display as shown by the “Navigation and Measurement Controls.” Accordingly, during the procedure, a surgeon can simultaneously receive surgical assistance and imaging assistance, increasing the likelihood that the procedure will run smoothly.

The data may be processed into any number of different displays, such as two, three, four, five, 10, etc. displays. In an exemplary embodiment, there are three types of displays. Those displays include real-time image display (approximating as closely as possible to the image currently being acquired by the device inside the patient); image display at a fixed rate (such as 5, 15 or 30 frames per second so that the viewer can appreciate each and every image); and a paused image. Typically, the paused image may be used to make analytical measurements about the lumen.

In the example user interface shown above, the longitudinal view controls are linked to a tomographic display that can be used for either fixed rate display or paused image display. The user determines which behavior is used through controls such as a play/pause button. One embodiment may offer an additional control to switch to real-time image display. In one embodiment, controls and indicators related to workflow are shown in a separate area to avoid confusion by the multiple users interacting with the system. In many cases, key aspects of the workflow are controlled by the user operating the handheld unit.

In some embodiments of the present invention, the tomographic images may be displayed on multiple screens or devices, each having a different behavior. For example, real-time images may be presented to the clinical user at the bedside. At the same time, another user who may even be located in another room may be seeing a fixed rate display, and a third user may be seeing a paused image display on which they are creating a measurement.

Some imaging devices are capable of acquiring images too quickly to be displayed to the user at their preferred rate for a fixed rate display (e.g. 30 frames per second). Thus, displays with this behavior will necessarily lag behind the images being acquired by the device and this lag will increase throughout acquisition.

In certain embodiments, the system includes buffering mechanisms such as random-access memory, high-speed disk storage and retrieval, and may also include network connections to accommodate the asynchronous display of information conceived for fixed-rate and paused image displays. The same mechanisms may be used to realize simultaneous display of different types of displays on different devices.

In certain embodiments, systems of the invention present only certain types of displays on certain devices while in certain workflow states (e.g. during acquisition of new image data only real-time may be displayed on a device in the operating room, while fixed-rate and paused images are not shown until acquisition has concluded; however a user in the control room may be able to switch between all three display types). Systems of the invention also allow for textually labeling the type of image data being displayed, and/or color-coding the image data or the background over which the image data is displayed.

Aspects of the invention are implemented using a rules-based approach that is carried out by: specifying a set of roles, workflow states and display rules within the system; determining which device fits a particular role and identifying it as such to the system; and performing the specified rules to present appropriate data to the appropriate device.

For example, a system may have a single set of workflow states, a bedside screen including one role-rule combination, and a control room screen including another role-rule combination. The installation process identifies which physical device such as an LCD monitor should receive a bedside screen and which device receives a control room screen. If a third physical device is available and is located in the control room it may also present a bedside screen so that the control room user can simultaneously see both their own view and the view of the physician user.

If a clinical user in the operating room wishes to choose between viewing real-time and fixed-rate image displays, this can be accomplished by ensuring that such a user is presented with a display that clearly indicates the nature of information being shown.

One exemplary embodiment is shown in FIG. 3. By displaying a longitudinal view with a current-frame indicator it is immediately apparent to the user which frame is being viewed, and whether such a frame is real-time or delayed. Furthermore, by presenting the accumulation of image data in real time within the longitudinal view the system complies with regulatory and safety requirements to indicate the emission of energy associated with imaging, regardless of which image display type is in use.

Systems and methods of the invention may be accept data from any intravascular imaging device. Exemplary devices include intravascular ultrasound (IVUS) devices and an optical coherence tomography (OCT) devices. With such devices, the data accepted by the system is IVUS data or OCT data. In one embodiment, the intravascular device is an IVUS device and the data is IVUS data. IVUS catheters and processing of IVUS data are described for example in Yock, U.S. Pat. Nos. 4,794,931, 5,000,185, and 5,313,949; Sieben et al., U.S. Pat. Nos. 5,243,988, and 5,353,798; Crowley et al., U.S. Pat. No. 4,951,677; Pomeranz, U.S. Pat. No. 5,095,911, Griffith et al., U.S. Pat. No. 4,841,977, Maroney et al., U.S. Pat. No. 5,373,849, Born et al., U.S. Pat. No. 5,176,141, Lancee et al., U.S. Pat. No. 5,240,003, Lancee et al., U.S. Pat. No. 5,375,602, Gardineer et at., U.S. Pat. No. 5,373,845, Seward et al., Mayo Clinic Proceedings 71(7):629-635 (1996), Packer et al., Cardiostim Conference 833 (1994), “Ultrasound Cardioscopy,” Eur. J.C.P.E. 4(2):193 (June 1994), Eberle et al., U.S. Pat. No. 5,453,575, Eberle et al., U.S. Pat. No. 5,368,037, Eberle et at., U.S. Pat. No. 5,183,048, Eberle et al., U.S. Pat. No. 5,167,233, Eberle et at., U.S. Pat. No. 4,917,097, Eberle et at., U.S. Pat. No. 5,135,486, and other references well known in the art relating to intraluminal ultrasound devices and modalities.

In another embodiment, the intravascular device is an OCT catheter and the data is OCT data. OCT is a medical imaging methodology using a miniaturized near infrared light-emitting probe. As an optical signal acquisition and processing method, it captures micrometer-resolution, three-dimensional images from within optical scattering media (e.g., biological tissue). Recently it has also begun to be used in interventional cardiology to help diagnose coronary artery disease. OCT allows the application of interferometric technology to see from inside, for example, blood vessels, visualizing the endothelium (inner wall) of blood vessels in living individuals.

OCT systems and methods are generally described in Castella et al., U.S. Pat. No. 8,108,030, Milner et al., U.S. Patent Application Publication No. 2011/0152771, Condit et al., U.S. Patent Application Publication No. 2010/0220334, Castella et al., U.S. Patent Application Publication No. 2009/0043191, Milner et al., U.S. Patent Application Publication No. 2008/0291463, and Kemp, N., U.S. Patent Application Publication No. 2008/0180683, the content of each of which is incorporated by reference in its entirety.

In some embodiments, a user interacts with a visual interface to view images from the imaging system. Input from a user (e.g., parameters or a selection) are received by a processor in an electronic device. The selection can be rendered into a visible display. An exemplary system including an electronic device is illustrated in FIG. 4. As shown in FIG. 4, a sensor engine 859 communicates with host workstation 433 as well as optionally server 413 over network 409. The data acquisition element 855 (DAQ) of the sensor engine receives sensor data from one or more sensors. In some embodiments, an operator uses computer 449 or terminal 467 to control system 400 or to receive images. An image may be displayed using an I/O 454, 437, or 471, which may include a monitor. Any I/O may include a keyboard, mouse or touchscreen to communicate with any of processor 421, 459, 441, or 475, for example, to cause data to be stored in any tangible, nontransitory memory 463, 445, 479, or 429. Server 413 generally includes an interface module 425 to effectuate communication over network 409 or write data to data file 417.

Processors suitable for the execution of computer program include, by way of example, both general and special purpose microprocessors, and any one or more processor of any kind of digital computer. Generally, a processor will receive instructions and data from a read-only memory or a random access memory or both. The essential elements of computer are a processor for executing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. Information carriers suitable for embodying computer program instructions and data include all forms of non-volatile memory, including by way of example semiconductor memory devices, (e.g., EPROM, EEPROM, solid state drive (SSD), and flash memory devices); magnetic disks, (e.g., internal hard disks or removable disks); magneto-optical disks; and optical disks (e.g., CD and DVD disks). The processor and the memory can be supplemented by, or incorporated in, special purpose logic circuitry.

To provide for interaction with a user, the subject matter described herein can be implemented on a computer having an I/O device, e.g., a CRT, LCD, LED, or projection device for displaying information to the user and an input or output device such as a keyboard and a pointing device, (e.g., a mouse or a trackball), by which the user can provide input to the computer. Other kinds of devices can be used to provide for interaction with a user as well. For example, feedback provided to the user can be any form of sensory feedback, (e.g., visual feedback, auditory feedback, or tactile feedback), and input from the user can be received in any form, including acoustic, speech, or tactile input.

The subject matter described herein can be implemented in a computing system that includes a back-end component (e.g., a data server 413), a middleware component (e.g., an application server), or a front-end component (e.g., a client computer 449 having a graphical user interface 454 or a web browser through which a user can interact with an implementation of the subject matter described herein), or any combination of such back-end, middleware, and front-end components. The components of the system can be interconnected through network 409 by any form or medium of digital data communication, e.g., a communication network. Examples of communication networks include cell network (e.g., 3G or 4G), a local area network (LAN), and a wide area network (WAN), e.g., the Internet.

The subject matter described herein can be implemented as one or more computer program products, such as one or more computer programs tangibly embodied in an information carrier (e.g., in a non-transitory computer-readable medium) for execution by, or to control the operation of, data processing apparatus (e.g., a programmable processor, a computer, or multiple computers). A computer program (also known as a program, software, software application, app, macro, or code) can be written in any form of programming language, including compiled or interpreted languages (e.g., C, C++, Perl), and it can be deployed in any form, including as a stand-alone program or as a module, component, subroutine, or other unit suitable for use in a computing environment. Systems and methods of the invention can include instructions written in any suitable programming language known in the art, including, without limitation, C, C++, Perl, Java, ActiveX, HTML5, Visual Basic, or JavaScript.

A computer program does not necessarily correspond to a file. A program can be stored in a portion of file 417 that holds other programs or data, in a single file dedicated to the program in question, or in multiple coordinated files (e.g., files that store one or more modules, sub-programs, or portions of code). A computer program can be deployed to be executed on one computer or on multiple computers at one site or distributed across multiple sites and interconnected by a communication network.

A file can be a digital file, for example, stored on a hard drive, SSD, CD, or other tangible, non-transitory medium. A file can be sent from one device to another over network 409 (e.g., as packets being sent from a server to a client, for example, through a Network Interface Card, modem, wireless card, or similar).

Writing a file according to the invention involves transforming a tangible, non-transitory computer-readable medium, for example, by adding, removing, or rearranging particles (e.g., with a net charge or dipole moment into patterns of magnetization by read/write heads), the patterns then representing new collocations of information about objective physical phenomena desired by, and useful to, the user. In some embodiments, writing involves a physical transformation of material in tangible, non-transitory computer readable media (e.g., with certain optical properties so that optical read/write devices can then read the new and useful collocation of information, e.g., burning a CD-ROM). In some embodiments, writing a file includes transforming a physical flash memory apparatus such as NAND flash memory device and storing information by transforming physical elements in an array of memory cells made from floating-gate transistors. Methods of writing a file are well-known in the art and, for example, can be invoked manually or automatically by a program or by a save command from software or a write command from a programming language.

### Incorporation by Reference

References and citations to other documents, such as patents, patent applications, patent publications, journals, books, papers, web contents, have been made throughout this disclosure. All such documents are hereby incorporated herein by reference in their entirety for all purposes.

### Equivalents

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

What is claimed is:

1. A system for managing medical image data for multiple users, the system comprising:
a central processing unit (CPU); and
storage coupled to said CPU for storing instructions that when executed by the CPU cause the CPU to:
accept as input, real-time image data representative of an inside of a lumen from an intravascular imaging device;
associate the data with a type of device used to acquire the data;
process the data into a plurality of different displays;
determine which user should see which type of display; and
provide as an output, the proper display to each user.

2. The system according to claim 1, wherein there are three types of displays.

3. The system according to claim 2, wherein the three types of displays are (1) real-time image display; (2) image display at a fixed rate; and (3) a paused image.

4. The system according to claim 3, wherein the paused image is used to make analytical measurements about the lumen.

5. The system according to claim 1, wherein multiple users are provided a display simultaneously.

6. The system according to claim 1, wherein a single user is provided more than one display.

7. The system according to claim 1, wherein the intravascular imaging device is an intravascular ultrasound (IVUS) device or an optical coherence tomography (OCT) device.

8. The system according to claim 7, wherein the image data is IVUS data or OCT data.

9. The system according to claim 1, wherein the CPU is further caused to label the type of data to be displayed.

10. The system according to claim 1, wherein the CPU is further caused to color-code the image data or the background over which the image is displayed.

11. A method for managing medical image data for multiple users, the method comprising:
receiving in real-time, image data representative of an inside of a lumen from an intravascular imaging device;
associating the data with the type of device used to acquire the data;
processing the data into a plurality of different displays;
determining which user should see which type of display; and
providing as an output, the proper display to each user.

12. The method according to claim 1, wherein the there are three types of displays.

13. The method according to claim 12, wherein the three types of displays are (1) real-time image display; (2) image display at a fixed rate; and (3) a paused image.

14. The method according to claim 13, wherein the paused image is used to make analytical measurements about the lumen.

15. The method according to claim 11, wherein multiple users are provided a display simultaneously.

16. The method according to claim 11, wherein a single user is provided more than one display.

17. The method according to claim 11, wherein the intravascular imaging device is an intravascular ultrasound (IVUS) device or an optical coherence tomography (OCT) device.

18. The method according to claim 17, wherein the image data is IVUS data or OCT data.

19. The method according to claim 11, wherein the CPU is further caused to label the type of data to be displayed.

20. The method according to claim 11, wherein the CPU is further caused to color-code the image data or the background over which the image is displayed.
